(19) United States
(12) Patent Application Publication
(10) Pub. No.: US 2021/0107866 A1
(43) Pub. Date: Apr. 15, 2021

(54) UREA MANUFACTURING METHOD

(71) Applicant: Toyo Engineering Corporation, Chiyoda-ku, Tokyo (JP)

(72) Inventors: Eiki NAGASHIMA, Narashino-shi, Chiba (JP); Masashi TAKAHASHI, Narashino-shi, Chiba (JP)

(21) Appl. No.: 17/046,432

(22) PCT Filed: Apr. 3, 2019
(86) PCT No.: PCT/JP2019/014847

§ 371 Date: Oct. 9, 2020

(30) Foreign Application Priority Data
Apr. 13, 2018 (JP) 2018-077244

(51) Int. Cl. C07C 273/04; B01J 19/02; B01J 19/00

(52) U.S. Cl. CPC C07C 273/04; C07C 275/02; B01J 19/0053; B01J 19/02

(57) ABSTRACT

[Problem to be solved] To provide a urea manufacturing method capable of inhibiting corrosion of a urea plant and enhancing a reaction yield.

[Solution] In a urea manufacturing plant, a method for manufacturing urea from manufacturing raw materials including NH₃ and CO₂, wherein the urea manufacturing plant includes a plurality of processing units including a reactor, a stripper and a condenser, and a plurality of lines, and the inner wall surfaces of the plurality of processing units and the plurality of lines are made of a stainless steel and at least some of the plurality of lines is made of an austenitic stainless steel, the urea manufacturing method including: forming a passivation film on the inner wall surfaces of the plurality of processing units and the plurality of lines by supplying CO₂ of the manufacturing raw material with added oxygen; continuously measuring a wall thickness of the line made of the austenitic stainless steel; and adjusting a supply amount of the oxygen in response to a measurement value of the wall thickness to control a corrosion rate and a reaction yield of urea.

### FIELD OF THE INVENTION

The present invention relates to a urea manufacturing method.

### BACKGROUND OF THE INVENTION

In urea manufacturing plants, highly corrosive ammonium carbamate is produced as an intermediate during processes of synthesizing urea from ammonia and carbon dioxide. Therefore, corrosion resistance is required of various processing units and lines of the plants.

JP-B 3987607 discloses inventions of a urea synthesis method and a urea synthesis apparatus and explains that corrosion preventive air is introduced into a condenser, a synthesis column and a stripper (see paragraphs 0028, 0046, 0055 and 0070).

WO-A 2014-192823 discloses an invention of a urea synthesis method. It explains that, in a urea synthesis apparatus for performing a urea synthesis method, at least some of portions at which a urea synthesis tower A, a stripper B and a condenser C, and piping connecting them come in contact with corrosive fluids may be made of an austenite-ferrite duplex stainless steel of a particular composition, and in addition, in piping, valves and the like, S31603 series general-purpose stainless steel may also be used according to corrosion environments. WO-A 2014-192823 explains that an amount of corrosion preventive oxygen fed may be reduced, inert gases are reduced, and a reaction yield is enhanced (Effects of the Invention).

### SUMMARY OF THE INVENTION

An object of the present invention is to provide a urea manufacturing method capable of enhancing a reaction yield of urea by inhibiting corrosion of processing units and lines of a urea plant when manufacturing urea by the plant.

The present invention provides a method for manufacturing urea from manufacturing raw materials including ammonia and carbon dioxide in a urea manufacturing plant,

wherein the urea manufacturing plant includes a plurality of processing units including a reactor, a stripper and a condenser, and a plurality of lines connecting the plurality of processing units, and

the inner wall surfaces of the plurality of processing units and the plurality of lines are made of a stainless steel, and at least some of the plurality of lines is made of an austenitic stainless steel,

the urea manufacturing method including: forming a passivation film on the inner wall surfaces of the plurality of processing units and the plurality of lines by supplying carbon dioxide of the manufacturing raw material with added oxygen; continuously measuring a wall thickness of the line made of the austenitic stainless steel; and adjusting a supply amount of the oxygen in response to a measurement value of the wall thickness to control a corrosion rate and a reaction yield of urea (a control method (A)).

In addition, the present invention provides a method for manufacturing urea from manufacturing raw materials including ammonia and carbon dioxide in a urea manufacturing plant,

wherein the urea manufacturing plant includes a plurality of processing units including a reactor, a stripper and a condenser, and a plurality of lines connecting the plurality of processing units, and

the inner wall surfaces of the plurality of processing units and the plurality of lines are made of a stainless steel, and at least some of the plurality of lines is made of an austenitic stainless steel,

the urea manufacturing method including: forming a passivation film on the inner wall surfaces of the plurality of processing units and the plurality of lines by supplying carbon dioxide of the manufacturing raw material with added oxygen; measuring a concentration of iron, chromium or nickel dissolved in urea or ammonia and an operating temperature; and adjusting a supply amount of the oxygen in response to measurement values of the concentration and the operating temperature to control a corrosion rate and a reaction yield of urea (a control method (B)).

Further, the present invention provides a method for manufacturing urea from manufacturing raw materials including ammonia and carbon dioxide in a urea manufacturing plant,

wherein the urea manufacturing plant includes a plurality of processing units including:

a reactor to produce a urea synthesis liquid using carbon dioxide and ammonia as raw materials;

a stripper to decompose ammonium carbamate and separate a mixed gas including ammonia and carbon dioxide from the urea synthesis liquid produced in the reactor by heating the urea synthesis liquid; and

a condenser to condense at least a portion of the mixed gas obtained in the stripper by absorption onto an absorption medium and to generate a low-pressure steam using heat generated at the time of the condensation, and

a plurality of lines connecting the plurality of processing units, and

the inner wall surfaces of the plurality of processing units and the plurality of lines are made of a stainless steel, and at least some of the plurality of lines is made of an austenitic stainless steel,

the urea manufacturing method including performing any one or any two or three of the following control methods (A) to (C):

(A) a control method in which, in the urea manufacturing method, a passivation film is formed on the inner wall surfaces of the plurality of processing units and the plurality of lines by supplying carbon dioxide of the manufacturing raw material with added oxygen, a wall thickness of the line made of the austenitic stainless steel is continuously measured, and a supply amount of the oxygen is adjusted in response to a measurement value of the wall thickness to control a corrosion rate and a reaction yield of urea;

(B) a control method in which a concentration of iron, chromium or nickel dissolved in urea or ammonia and an operating temperature are measured, and a supply amount of the oxygen is adjusted in response to measurement values of the concentration and the operating temperature to control a corrosion rate and a reaction yield of urea; and

(C) a control method in which operating pressures of the plurality of processing units and their respective operating temperatures, a flow rate of carbon dioxide introduced as the raw material, an oxygen amount in the raw material carbon dioxide and a flow rate of ammonia introduced as the raw material are measured to calculate respective corrosion rates of the plurality of processing units and corrosion rates of the plurality of lines connecting the plurality of processing units, and a supply amount of the oxygen is adjusted thereby to control a corrosion rate and a reaction yield of urea.

According to the urea manufacturing method of the present invention, corrosion of processing units and lines of urea manufacturing plants during urea manufacturing processes can be inhibited and a yield of urea can thereby be maintained.

### BRIEF DESCRIPTION OF DRAWINGS

FIG. 1 A schematic view showing a urea manufacturing flow in a urea manufacturing plant.

FIG. 2 A diagram for illustrating one embodiment of a urea manufacturing method using the urea manufacturing plant.

FIG. 3 A plot indicating a difference in corrosion rates between a pipe on which a passivation film layer is formed and a pipe on which no passivation film layer is formed in Example 1.

### EMBODIMENTS OF THE INVENTION

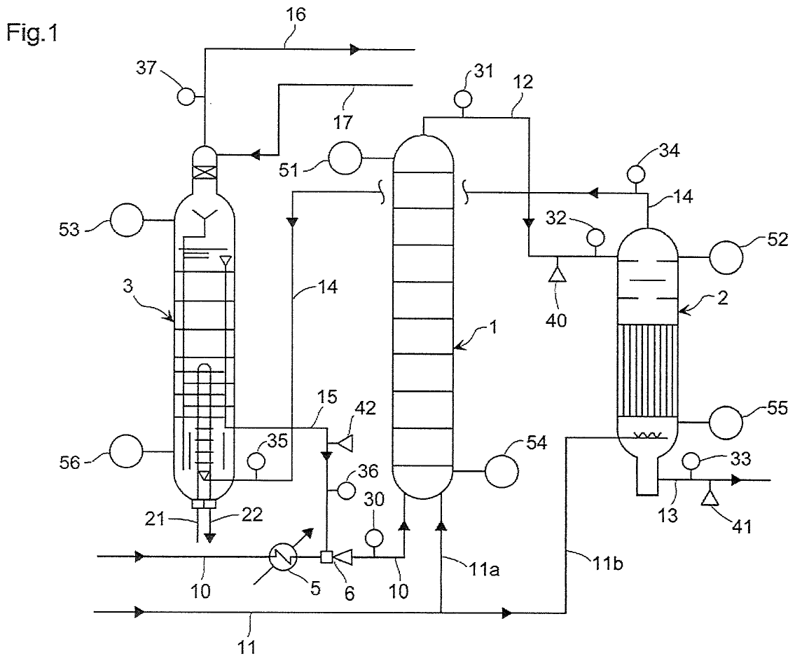

A urea manufacturing method of the present invention is explained referencing FIG. 1. A urea manufacturing plant shown in FIG. 1 is one embodiment for performing the urea manufacturing method of the present invention and it is not limited thereto.

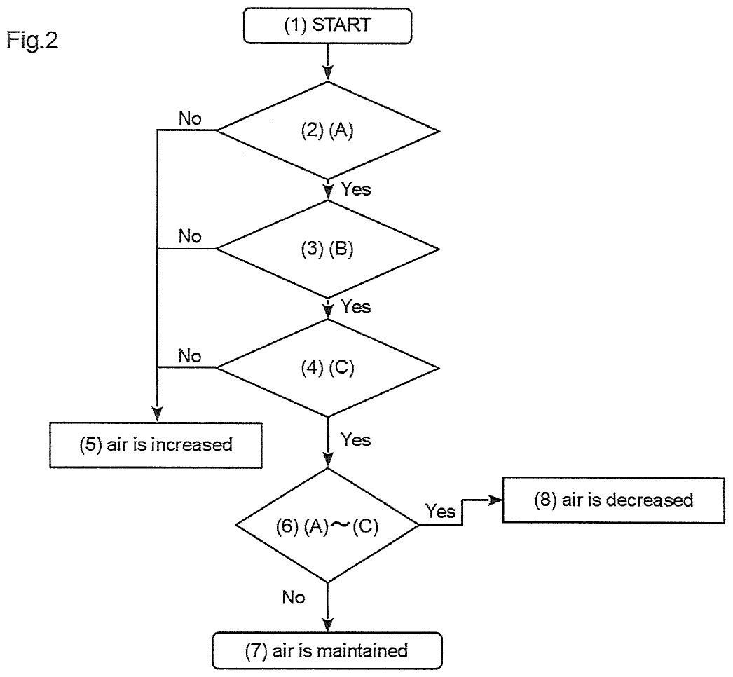

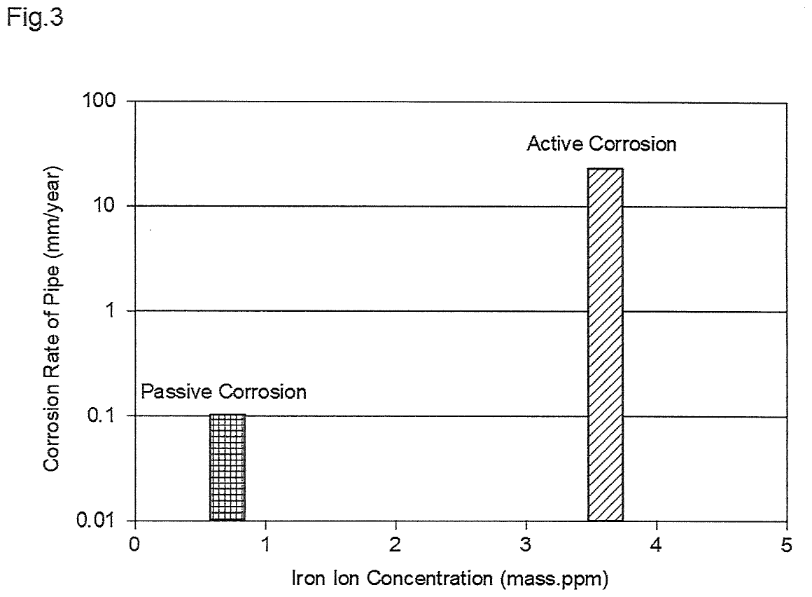

In addition, a urea manufacturing flow in the urea plant shown in FIG. 1 itself is publicly known and substantially the same as, for example, those shown in FIG. 3 of JP-B 3987607 and FIG. 2 of WO-A 2014-192823. A reactor 1, a stripper 2, a condenser 3, a heat exchanger 5 and an ejector 6 shown in FIG. 1 are the same as a urea synthesis column A, a stripper C, a condenser B (including a scrubber F), a heat exchanger D and an ejector G shown in FIG. 3 of JP-B 3987607, respectively.

A feature of the urea manufacturing method of the present invention is to control, for example, when urea is manufactured in the urea manufacturing plant shown in FIG. 1, a corrosion rate and a reaction yield of urea by adjusting a supply amount of oxygen in response to certain measurement values, and the manufacturing method including specific manufacturing processes and reaction conditions is not particularly limited.

In the urea manufacturing method of the present invention, urea may also be manufactured by, for example, a manufacturing method using a urea manufacturing plant shown in FIG. 3 of JP-B 3987607 and using the same manufacturing processes and conditions as a manufacturing method described in paragraphs 0052 to 0062 or Example 3, or a manufacturing method using the same manufacturing processes and conditions as a manufacturing method described in paragraphs 0040 to 0048 or 0060 of WO-A 2014-192823.

In the manufacturing flow example shown in FIG. 1, ammonia is supplied from an ammonia supply line 10 to the bottom of the reactor 1, and in parallel therewith, carbon dioxide is supplied from carbon dioxide supply lines 11 and 11a to the bottom of the reactor 1, and they are reacted inside the reactor 1 and a gas-liquid mixture including urea is thereby obtained. The reactor 1 is a unit to produce a urea synthesis liquid using carbon dioxide and ammonia as raw materials.

The reactor 1 is made of, for example, carbon steel, and on a portion corresponding to the inner wall surface, a lining layer made of duplex stainless steel is formed. Therefore, the wall thickness of the reactor 1 cannot be measured with ultrasonic wall thickness gauges from the outside.

In the gas-liquid mixture obtained in the reactor 1, urea, ammonium carbamate which is a reaction intermediate, water and unreacted ammonia are present as the liquid phase, and some unreacted ammonia, unreacted carbon dioxide and inert gases are present as the gas phase. The inert gases are impurities such as air (oxygen) supplied for a corrosion protection purpose and hydrogen included in the raw material carbon dioxide.

Reaction conditions in the reactor 1 may be the same as those in the case of using the urea manufacturing plant shown in FIG. 3 of JP-B 3987607 as mentioned above, and for example, it is preferable that a pressure be 130 to 250 bar (13,000 to 25,000 kPa), an N/C (a molar ratio of ammonia and carbon dioxide) be 3.5 to 5.0, an H/C (a molar ratio of water and carbon dioxide) be 1.0 or less, a residence time be 10 to 40 minutes, and a temperature be 180 to 200° C.

When carbon dioxide is supplied to the reactor 1, it is pressurized by a compressor (which is connected to the carbon dioxide supply lines 11 and 11a, although not illustrated) as well as mixed with an adjustment amount of oxygen. The oxygen may be pure oxygen or air. When air is used, the air is preferably supplied through an air filter or the like.

Ammonia is, on its way to being supplied from the ammonia supply line 10 to the reactor 1, preheated to approximately 70 to 90° C. via the heat exchanger 5 and thereafter supplied to the reactor 1 together with ammonia collected from the condenser 3 by the ejector 6.

The gas-liquid mixture obtained in the reactor 1 is delivered through a gas-liquid mixture line 12 to the top of the stripper 2. The stripper 2 is a unit to separate the mixed gas including unreacted ammonia and unreacted carbon dioxide from the urea synthesis liquid produced in the reactor 1 by heating the urea synthesis liquid.

The stripper 2 is made of, for example, carbon steel, and on a portion corresponding to the inner wall surface, a lining layer made of duplex stainless steel is formed. Therefore, the wall thickness of the stripper 2 cannot be measured with ultrasonic wall thickness gauges from the outside.

From the bottom of the stripper 2, a carbon dioxide gas serving as a stripping agent is supplied from the carbon dioxide supply lines 11 and lib. The stripper 2 is heated by a heater not illustrated so that the internal temperature can be increased.

Operating conditions in the stripper 2 may be the same as those in the case of using the urea manufacturing plant shown in FIG. 3 of JP-B 3987607 as mentioned above, and for example, it is preferable that a pressure be 130 to 250 bar (13,000 to 25,000 kPa) and preferably 140 to 200 bar (14,000 to 20,000 kPa), and a temperature be 160 to 200° C.

In the stripper 2, due to the heating and the introduction of carbon dioxide serving as a stripping agent, ammonium carbamate in the gas-liquid mixture decomposes into ammonia and carbon dioxide to be delivered through a returned gas line 14 to the bottom of the condenser 3 as a high-temperature mixed gas of unreacted ammonia, carbon dioxide, inert gases and water (vapor).

Urea, a trace amount of undecomposed ammonium carbamate, unseparated ammonia, carbon dioxide and the like in the gas-liquid mixture are collected through a urea collecting line 13 at the bottom of the stripper 2. The urea collected through the urea collecting line 13 is further subjected to a purification process in a subsequent step (a low-pressure decomposition step) and thereby enhanced in purity. The trace amount of residual ammonium carbamate is subjected to a decomposition process and thereby becomes a low-temperature recycle liquid including ammonia and carbon dioxide (also including unreacted ammonia and carbon dioxide) and is delivered through a recycle line 17 to the top of the condenser 3 (scrubber) as an absorption medium.

The condenser 3 is a unit to condense at least a portion of the mixed gas obtained in the stripper 2 by absorption onto the absorption medium and to generate a low-pressure steam using heat generated at the time of the condensation. Ammonia included in the mixed gas of a high-temperature state supplied to the bottom of the condenser 3 is cooled and condensed, and thereafter delivered through a down pipe 15 to the raw material ammonia supply line 10 by a suctioning function of the ejector 6, and recycled as a urea manufacturing material.

Some of the ammonia, carbon dioxide and water (vapor) accompanying the inert gases of a high-temperature state supplied to the bottom of the condenser 3 come in contact with the absorption medium during a process of being cooled and discharged as a low-temperature gas through an exhaust line 16, and the ammonia and the carbon dioxide are thereby absorbed and removed, and the inert gases are discharged through the exhaust line 16.

The condenser 3 is made of, for example, carbon steel, and on a portion corresponding to the inner wall surface, a lining layer made of duplex stainless steel is formed. Therefore, the wall thickness of the condenser 3 cannot be measured with ultrasonic wall thickness gauges from the outside.

Cooling water is introduced from a cooling water line 21 into the condenser 3 and heat-exchanged and vaporized therewithin into vapor, which is gathered through a vapor line 22 and recycled as high-temperature vapor. Operating conditions in the condenser 3 may be the same as those in the case of using the urea manufacturing plant shown in FIG. 3 of JP-B 3987607 as mentioned above, and for example, it is preferable that a pressure be 140 to 250 bar (14,000 to 25,000 kPa), a temperature be 130 to 250° C. (preferably 170 to 190° C.), an N/C be 2.5 to 3.5, an H/C be 1.0 or less, and a residence time be 10 to 30 minutes.

For each line mentioned above, a pipe of an austenitic stainless steel (single phase) or a pipe of a duplex stainless steel (austenite-ferrite duplex stainless steel) may be used. However, in the example of the urea plant shown in FIG. 1, each line with any of wall thickness measurement parts 30 to 37 by ultrasonic wall thickness gauges is made of the pipe of the austenitic stainless steel.

As the austenitic stainless steel, for example, S31603 (316L SS) may be used, and as the duplex stainless steel, for example, a 25Cr duplex stainless steel (S31260) or a 28Cr duplex stainless steel (S32808: DP28W) may be used. Since each line is made of a single material, the wall thickness can be measured with ultrasonic wall thickness gauges from the outside.

It is known that, during the urea manufacturing process in the urea manufacturing plant shown in FIG. 1, highly metal-corrosive ammonium carbamate, which is a byproduct of a reaction of ammonia and carbon dioxide, corrodes the inner wall surfaces of the reactor 1, the stripper 2 and the condenser 3 or corrodes the inner wall surface of each line.

In the manufacturing method of the present invention, contact of the stainless steels with ammonium carbamate is inhibited by forming a passivation film on the surfaces of the stainless steels by mixing oxygen into the raw material carbon dioxide, and the corrosion of the stainless steels is thereby inhibited. Note that the austenitic stainless steel has a property of requiring more oxygen compared to the duplex stainless steels in order to form the passivation film. However, a too-high oxygen concentration in the raw material carbon dioxide cannot fully increase temperatures inside the reactor 1 and inside the condenser 3 and cannot increase reaction rates either and therefore causes a reduction in a reaction yield of urea (a reduction in a yield), and a too-low oxygen concentration excessively promotes the corrosion of the stainless steels.

Note that FIG. 5 of WO-A 2014-192823 indicates a relation between an oxygen concentration in a gas phase (the horizontal axis) and a corrosion rate (the vertical axis). It suggests that, for the austenitic stainless steel (S31603), a passivation film is hard to form compared to the 25Cr duplex stainless steel (S31260) and the 28Cr duplex stainless steel (S32808), so that, when the oxygen concentration is low, the corrosion rate becomes larger, and when the oxygen concentration becomes higher, the corrosion rate becomes smaller because a passivation film is formed on any of the stainless steels.

In the manufacturing method of the present invention, it is preferable that any one or any two or three of the following control methods (A) to (C) be performed.

### Control Method (A)

A control method (A) is a control method in which, in the urea manufacturing method, a passivation film is formed on the inner wall surfaces of the plurality of processing units (including the reactor 1, the stripper 2 and the condenser 3) and the plurality of lines by supplying carbon dioxide of a manufacturing raw material with added oxygen, a wall thickness of a line made of an austenitic stainless steel is continuously measured, and a supply amount of the oxygen is adjusted in response to a measurement value of the wall thickness to control a corrosion rate and a reaction yield of urea.

In the example of the urea manufacturing plant shown in FIG. 1, a thickness t2 of each line during operation is continuously measured with ultrasonic wall thickness gauges, for example, at the wall thickness measurement parts 30 to 37, thereby determining a corrosion rate s (mm/year) from the following formula: s=(t1−t2)/(operating time) (t1 represents an initial thickness of each line at the wall thickness measurement parts 30 to 37 before operation).

The initial thickness t1 of each line is known (a measurement value or a specification value) and the corrosion rate s is obtained by dividing a difference between the initial thickness t1 and the thickness t2 of each line after operation by an operating time. Therefore, changes in the corrosion rate s can be continuously checked by continuously measuring the thickness t2 of each line during operation.

Accordingly, as an example of the control method (A), when the corrosion rate s becomes too high, the oxygen supply amount (when air is used, an air amount in terms of an oxygen amount) is increased, and when the corrosion rate s is sufficiently small, the oxygen supply amount is decreased. This makes it possible to inhibit variations of increase and decrease in the reaction yield of urea to be as small as possible so that urea can be manufactured at a stable reaction yield.

The corrosion rate s in each line at the time of operating the urea manufacturing plant shown in FIG. 1 is preferably controlled to be 0.2 mm/year or less and more preferably controlled to be 0.15 mm/year or less in terms of the relation between s and the reaction yield of urea.

### Control Method (B)

A control method (B) is a control method in which a concentration of iron, chromium or nickel dissolved in urea or ammonia and an operating temperature are measured, and a supply amount of the oxygen is adjusted in response to measurement values of the concentration and the operating temperature to control a corrosion rate and a reaction yield of urea.

In the example of the urea manufacturing plant shown in FIG. 1, sampling can be performed, for example, at sampling positions 40 to 42. At the sampling position 40, along with sampling of, for example, the gas-liquid mixture including urea, ammonium carbamate which is a reaction intermediate and unreacted gases (ammonia and carbon dioxide) flowing through the gas-liquid mixture line 12, the temperature is measured, and each ion concentration of iron, chromium or nickel in the sample is thereafter measured.

At the sampling position 41, along with sampling of, for example, urea, a trace amount of ammonium carbamate and the like flowing through the urea collecting line 13, the temperature is measured, and the respective ion concentrations of iron, chromium and nickel in the sample are thereafter measured.

At the sampling position 42, along with sampling of, for example, a liquid including ammonia flowing through the down pipe 15, the temperature is measured, and each ion concentration of iron, chromium or nickel in the sample is thereafter measured.

When a result of the measurement is that the respective ion concentrations of iron, chromium and nickel in the sample are high, it is assumed that the formation of the passivation film is insufficient and the corrosion is in progress, and when a result of the measurement is that each ion concentration of iron, chromium or nickel in the sample is low, it is assumed that the formation of the passivation film is sufficient and the corrosion is not in progress. The ion of iron, chromium or nickel to be measured may be any one of, or a combination of any two of, or all the three of them. In addition, as a result of the measurement, high temperatures at the sampling positions suggest that the corrosion progress becomes fast, and low temperatures at the sampling position suggest that the corrosion progress becomes slow.

When the control method (B) is performed, it is preferable that the sampling be performed at a plurality of locations in the urea manufacturing plant and operating temperatures be measured at the plurality of sampling locations. The sampling locations (temperature measurement locations) are not particularly limited and a plurality of locations (preferably three or more locations) can be selected. For example, the outlet-side line of the reactor 1 (gas-liquid mixture line 12), the outlet-side line of the stripper 2 (urea collecting line 13) and the outlet-side line of the condenser 3 (down pipe 15) are preferable.

Note that temperatures inside the units, i.e., the reactor 1, the stripper 2 and the condenser 3 which are each near the sampling locations are also preferably measured as the operating temperatures. The temperature measurement may be performed with publicly-known thermometers such as thermocouples or temperature measuring resistors.

Thus, variations of increase and decrease in the reaction yield of urea can be inhibited so that urea can be manufactured at a stable reaction yield by performing any of the followings as the control method (B):

when the concentrations of iron, chromium and nickel are high and the temperatures at the sampling positions are high, the oxygen supply amount is increased to form a passivation film (a first form of the control method (B));

when the concentrations of iron, chromium and nickel are low and the temperatures at the sampling positions are low, the oxygen supply amount is decreased (a second form of the control method (B));

when the concentrations of iron, chromium and nickel are high and the temperatures at the sampling positions are low, the oxygen supply amount is increased (however, an increase amount is less than that of the first form) to form a passivation film (a third form of the control method (B)); and

when the concentrations of iron, chromium and nickel are low and the temperatures at the sampling positions are high, the oxygen supply amount is decreased (however, a decrease amount is less than that of the second form) (a fourth form of the control method (B)).

### Control Method (C)

A control method (C) is a control method in which operating pressures of the plurality of processing units (the reactor, the stripper and the condenser) and their respective operating temperatures, a flow rate of carbon dioxide introduced as a raw material, an oxygen amount in the raw material carbon dioxide and a flow rate of ammonia introduced as a raw material are measured to calculate respective corrosion rates of the plurality of processing units and corrosion rates of the plurality of lines connecting the plurality of processing units, and a supply amount of the oxygen is adjusted thereby to control a corrosion rate and a reaction yield of urea.

The operating temperature of the reactor 1 may be measured, for example, at an upper (preferably near the top) measurement part (measuring instrument) 51 or a lower measurement part (measuring instrument) 54 of the reactor 1. The operating temperature of the stripper 2 may be measured, for example, at an upper (preferably near the top) measurement part (measuring instrument) 52 or a lower measurement part (measuring instrument) 55 of the stripper 2. The operating temperature of the condenser 3 may be measured, for example, at an upper (preferably near the top) measurement part (measuring instrument) 53 or a lower measurement part (measuring instrument) 56 of the condenser 3.

The reactor 1, the stripper 2 and the condenser 3 are approximately the same in pressure. These pressures may be measured, for example, at the line 11b or at an ammonia injection line to the condenser 3 not illustrated.

The flow rate of carbon dioxide introduced as a raw material may be measured, for example, at the carbon dioxide supply lines 11 and 11a. When carbon dioxide is supplied to the reactor 1, it is pressurized by a compressor as well as mixed with an adjustment amount of oxygen. As such, an oxygen amount in the raw material carbon dioxide may be calculated, for example, from an amount of air introduced into the compressor. The flow rate of ammonia introduced as a raw material may be measured, for example, at the ammonia supply line 10.

The respective corrosion rates of the reactor 1, the stripper 2 and the condenser 3 and those of the plurality of lines (the gas-liquid mixture line 12, the returned gas line 14 and the down pipe 15) connecting the reactor 1, the stripper 2 and the condenser 3 may be determined as follows from the aforementioned measurement data, i.e., the operating temperatures, the operating pressures, the flow rate of carbon dioxide, the oxygen concentration in carbon dioxide and the flow rate of ammonia. They may be determined considering the followings on the basis of the relation between the measurement data and the corrosion rate in the control method (A): the higher the operating temperatures become, the larger the corrosion rates become; the higher the ammonium carbamate concentrations become, the larger the corrosion rates become; and the higher the oxygen concentration in carbon dioxide becomes, the smaller the corrosion rates become.

A further preferable embodiment of the urea manufacturing method of the present invention is explained referencing FIG. 2. In the embodiment shown in FIG. 2, the control methods (A), (B) and (C) are performed in this order.

In stage (1), urea manufacture, for example, according to the manufacturing flow illustrated in FIG. 1, is started. After the start of urea manufacture, the control methods (A) to (C) for controlling a corrosion rate and a reaction yield of urea by adjusting a supply amount of oxygen are performed.

In stage (2), whether to increase the supply amount of air (oxygen) in the raw material carbon dioxide or to maintain the current amount is determined by the control method (A). When the corrosion rate determined in the control method (A) is within acceptable values (Yes), the method proceeds to stage (3). When the corrosion rate determined in the control method (A) exceeds an acceptable value (No), the method proceeds to stage (5) in order to enhance corrosion protective effect, and the urea manufacture is continued while the supply amount of air (oxygen) in the raw material carbon dioxide is increased. In stage (2), if the method proceeds to stage (5) and the supply amount of air (oxygen) in the raw material carbon dioxide is increased, stage (3) or later is not performed.

In stage (3), whether to increase the supply amount of air (oxygen) in the raw material carbon dioxide or to maintain the current amount is determined by the control method (B). When the corrosion rate determined in the control method (B) is within acceptable values (Yes), the method proceeds to stage (4). When the corrosion rate determined in the control method (B) exceeds an acceptable value (No), the method proceeds to stage (5) in order to enhance corrosion protective effect, and the urea manufacture is continued while the supply amount of air (oxygen) in the raw material carbon dioxide is increased. In stage (3), if the method proceeds to stage (5) and the supply amount of air (oxygen) in the raw material carbon dioxide is increased, stage (4) or later is not performed.

In stage (4), whether to increase the supply amount of air (oxygen) in the raw material carbon dioxide or to maintain the current amount is determined by the control method (C). When the corrosion rate determined in the control method (C) is within acceptable values (Yes), the method proceeds to stage (5). When the corrosion rate determined in the control method (C) exceeds an acceptable value (No), the method proceeds to stage (5) in order to enhance corrosion protective effect, and the urea manufacture is continued while the supply amount of air (oxygen) in the raw material carbon dioxide is increased. In stage (4), if the method proceeds to stage (5) and the supply amount of air (oxygen) in the raw material carbon dioxide is increased, stage (6) or later is not performed.

In stage (6), whether to decrease the supply amount of air (oxygen) in the raw material carbon dioxide or to maintain the current amount is determined by evaluating the control methods (A) to (C) as a whole. When any of the corrosion rates determined in the control methods (A) to (C) is equal to or less than the acceptable value but approximate to the acceptable value (for example, more than 95% of the acceptable value of the corrosion rate), the method proceeds to stage (7) and the current supply amount of air (oxygen) in the raw material carbon dioxide is maintained. When the corrosion rates determined in the control methods (A) to (C) are all much less than the acceptable values (for example, equal to or less than 95% of the acceptable values of the corrosion rates), the method proceeds to stage (8) and the supply amount of air (oxygen) in the raw material carbon dioxide is decreased.

In addition to the respective embodiments mentioned above, the present invention also includes the following embodiments.

As the processing units such as the reactor 1, the stripper 2 and the condenser 3 in the urea manufacturing plant of the example shown in FIG. 1 are made of carbon steel and on portions corresponding to the inner wall surfaces, lining layers made of duplex stainless steel are formed, the wall thicknesses cannot be measured with ultrasonic wall thickness gauges from the outside. In addition, as the processing units such as the reactor 1, the stripper 2 and the condenser 3 are so high in temperature and pressure during operation that the inside cannot be observed either, corrosion states of the processing units cannot be checked directly during the operation of the urea manufacturing plant. On the other hand, each line shown in FIG. 1 is made of the single material stainless steel and the wall thickness can be measured with ultrasonic wall thickness gauges from the outside so that the corrosion state can be checked.

Thus, during the operation of the urea manufacturing plant shown in FIG. 1, the operation data such as the temperatures and the pressures of the processing units such as the reactor 1, the stripper 2 and the condenser 3 during operation and operating times thereof can be obtained, and along therewith, the wall thickness of each line (the wall thicknesses of the wall thickness measurement parts 30 to 37) can be measured and stored as relevant data. Further, the operation of the urea manufacturing plant shown in FIG. 1 can be ceased periodically, and the corrosion states of the lining layers made of duplex stainless steel inside the processing units such as the reactor 1, the stripper 2 and the condenser 3 can be observed and stored as data.

By comparing and evaluating the operation data such as the temperature, the pressure and the operating time of each processing unit, the wall thickness data of each line, and the observation data of the corrosion state of each processing unit with one another, the corrosion state inside each processing unit can be estimated from the wall thickness data of each line. This makes it possible to estimate the corrosion state inside each processing unit from change data of the wall thickness of each line while continuously operating the urea manufacturing plant. Therefore, without ceasing the operation of the urea manufacturing plant, a replacement timing or a maintenance timing of each processing unit can be ascertained, and a stable urea manufacturing operation can be performed.

Note that, while the present embodiment is suitable for a case of manufacturing urea while keeping the amount of oxygen (when air is used, air in terms of an oxygen amount) introduced into the urea manufacturing raw material constant instead of increasing or decreasing the oxygen supply amount (when air is used, an air amount in terms of an oxygen amount) as the aforementioned control methods (A) and (B), it can be performed in combination with one or both of the aforementioned control methods (A) and (B).

### EXAMPLES

### Example 1

Test pieces made of the stainless steels (28Cr duplex stainless steel; 532808, and austenitic stainless steel; 531603) were immersed respectively in urea liquids synthesized within an autoclave. In this state, oxygen was gradually introduced into the autoclave and oxygen amounts when passivation films were formed on the test pieces (Passive Corrosion) were measured. The test was conducted at a testing temperature of 195° C. The results are shown in FIG. 3.

As is evident from FIG. 3, for 531603, when the passivation film was formed (Passive Corrosion), a corrosion portion was as slight as 0.1 mm, whereas when the passivation film was insufficiently formed (Active Corrosion), the corrosion portion was much more than 10 mm. Note that it was able to be verified through the experiment that the oxygen amount required for the formation of the passivation film on S32808 was smaller compared to S31603.

From this result, it was verified that, in the urea manufacturing method of the present invention, the corrosion rate was able to be controlled as follows: a passivation film was formed on the inner wall surfaces of the plurality of processing units and the plurality of lines constituting the urea plant shown in FIG. 1; concentrations of iron, chromium and nickel dissolved in urea or ammonia and an operating temperature were measured; and a supply amount of oxygen was adjusted in response to measurement values of the concentrations and the operating temperature. Further, as it is a well-known fact that, in the urea manufacturing process, a large oxygen amount (air amount) reduces a reaction yield of urea, it was verified that, by adjusting the supply amount of oxygen, not only the corrosion rate, but also the reaction yield of urea was able to be controlled.

### Example 2

During the process of manufacturing urea according to the manufacturing flow of the urea manufacturing plant shown in FIG. 1, the following control methods (A), (B) and (C) were performed.

### Control Method (A)

60 days after start of urea manufacturing operation, the wall thickness (at the wall thickness measurement part 35) of the returned gas line 14 (an initial wall thickness: 23.01 mm) made of S31603 series general-purpose stainless steel (austenitic stainless steel) connecting the stripper 2 and the condenser 3 was measured with an ultrasonic wall thickness gauge (an ultrasonic thickness gauge of GE Sensing & Inspection Technologies Co., Ltd., a downsized, simply operated and high-performance ultrasonic thickness gauge DM5E series). A corrosion rate determined from a difference between the measured wall thickness and the initial wall thickness and the elapsed time was 0.12 mm/year. During a period from the start of operation to the time of measuring, a concentration of oxygen supplied into the raw material carbon dioxide had been 5500 ppm and an operating temperature (the average value) had been 183° C.

Based on the obtained corrosion rate, it was determined that a passivation film had been formed on the inner wall surface of the returned gas line 14. This means, in the embodiment shown in FIG. 2, that stage (2) is “Yes”. Accordingly, the method proceeds to stage (3).

### Control Method (B)

An iron concentration in a solution at an outlet of the stripper 2 (the sampling position 41) was 0.8 ppm and an operating temperature at that time was 171° C. Based on the obtained iron concentration, it was determined that a passivation film had been formed on the respective inner wall surfaces of the reactor 1, the gas-liquid mixture line 12 and the stripper 2 located upstream of the sampling position 41. This means, in the embodiment shown in FIG. 2, that stage (3) is “Yes”. Accordingly, the method proceeds to stage (4).

### Control Method (C)

Operating temperatures and operating pressures of measurement parts 51 to 53 were as follows:

measurement part 51: a temperature of 186° C., a pressure of 151 kg/cm²G;

measurement part 52: a temperature of 188° C., a pressure of 151 kg/cm²G; and

measurement part 53: a temperature of 180° C., a pressure of 151 kg/cm²G.

A flow rate of carbon dioxide (measured at the carbon dioxide supply lines 11 and 11a) was 45000 Nm³/h. An oxygen amount in the raw material carbon dioxide was 250 Nm³/h (calculated from an air amount introduced into the compressor). A flow rate of ammonia (measured at the ammonia supply line 10) was 69 t/h. Based on data including the measurement results above and the corrosion rate in the control method (A), a corrosion rate of each unit and each line was calculated as follows.

(i) the condenser 3 (the inner wall surface is made of S31603 series general-purpose stainless steel): 0.09 mm/year, temperature (180° C.)

(ii) the stripper 2 (the inner wall surface is made of duplex stainless steel): 0.10 mm/year, temperature (188° C.)

(iii) the reactor 1 (the inner wall surface is made of S31603 series general-purpose stainless steel): 0.14 mm/year, temperature (186° C.)

(iv) the returned gas line 14 from the stripper 2 to the condenser 3 (the inner wall surface is made of 531603 series general-purpose stainless steel): 0.16 mm/year, temperature (188° C.)

(v) the down pipe 15 from the condenser 3 to the reactor 1 (the inner wall surface is made of S31603 series general-purpose stainless steel): 0.09 mm/year, temperature (180° C.)

(vi) the gas-liquid mixture line 12 from the reactor 1 to the stripper 2 (the inner wall surface is made of 531603 series general-purpose stainless steel): 0.14 mm/year, temperature (186° C.)

In any of (i) to (vi), a concentration of oxygen supplied into the raw material carbon dioxide was 5525 ppm, and based on the obtained corrosion rates, it was determined that a passivation film had been formed on the inner surfaces of the respective units and those of the respective lines. This means, in the embodiment shown in FIG. 2, that stage (4) is “Yes”. Accordingly, the method proceeds to stage (6). Consequently, it was determined that the corrosion rates were less than the acceptable values and the oxygen amount was able to be decreased, and the oxygen concentration in the raw material carbon dioxide was decreased to 4500 ppm (stage (6)→stage (8) shown in FIG. 2).

### INDUSTRIAL APPLICABILITY

The urea manufacturing method of the present invention is capable of, when manufacturing urea by using publicly-known urea manufacturing plants, manufacturing urea in a reaction yield-efficient manner while extending lifetimes of the plants. Therefore, it can be applied as a manufacturing method capable of reducing a plant operating cost and a urea manufacturing cost.

### DESCRIPTION OF REFERENCE NUMERALS

1 reactor
2 stripper
3 condenser
5 heat exchanger
6 ejector
30-37 wall thickness measurement parts
40-42 sampling positions
51-56 temperature measurement parts

1. In a urea manufacturing plant, a method for manufacturing urea from manufacturing raw materials including ammonia and carbon dioxide,
wherein the urea manufacturing plant comprises a plurality of processing units including a reactor, a stripper and a condenser, and a plurality of lines connecting the plurality of processing units, and
the inner wall surfaces of the plurality of processing units and the plurality of lines are made of a stainless steel, and at least some of the plurality of lines is made of an austenitic stainless steel,
the urea manufacturing method comprising: forming a passivation film on the inner wall surfaces of the plurality of processing units and the plurality of lines by supplying carbon dioxide of the manufacturing raw material with added oxygen; continuously measuring a wall thickness of the line made of the austenitic stainless steel; and adjusting a supply amount of the oxygen in response to a measurement value of the wall thickness to control a corrosion rate and a reaction yield of urea.

2. In a urea manufacturing plant, a method for manufacturing urea from manufacturing raw materials including ammonia and carbon dioxide,
wherein the urea manufacturing plant comprises a plurality of processing units including a reactor, a stripper and a condenser, and a plurality of lines connecting the plurality of processing units, and
the inner wall surfaces of the plurality of processing units and the plurality of lines are made of a stainless steel, and at least some of the plurality of lines is made of an austenitic stainless steel,
the urea manufacturing method comprising: forming a passivation film on the inner wall surfaces of the plurality of processing units and the plurality of lines by supplying carbon dioxide of the manufacturing raw material with added oxygen; measuring a concentration of iron, chromium or nickel dissolved in urea or ammonia and an operating temperature; and adjusting a supply amount of the oxygen in response to measurement values of the concentration and the operating temperature to control a corrosion rate and a reaction yield of urea.

3. In a urea manufacturing plant, a method for manufacturing urea from manufacturing raw materials including ammonia and carbon dioxide,
wherein the urea manufacturing plant comprises a plurality of processing units including:
a reactor to produce a urea synthesis liquid using carbon dioxide and ammonia as raw materials;
a stripper to separate a mixed gas including unreacted ammonia and unreacted carbon dioxide from the urea synthesis liquid produced in the reactor by heating the urea synthesis liquid; and
a condenser to condense at least a portion of the mixed gas obtained in the stripper by absorption onto an absorption medium and to generate a low-pressure steam using heat generated at the time of the condensation, and a plurality of lines connecting the plurality of processing units, and
the inner wall surfaces of the plurality of processing units and the plurality of lines are made of a stainless steel, and at least some of the plurality of lines is made of an austenitic stainless steel,
the urea manufacturing method comprising performing any one or any two or three of the following control methods (A) to (C):
(A) a control method in which, in the urea manufacturing method, a passivation film is formed on the inner wall surfaces of the plurality of processing units and the plurality of lines by supplying carbon dioxide of the manufacturing raw material with added oxygen, a wall thickness of the line made of the austenitic stainless steel is continuously measured, and a supply amount of the oxygen is adjusted in response to a measurement value of the wall thickness to control a corrosion rate and a reaction yield of urea;
(B) a control method in which a concentration of iron, chromium or nickel dissolved in urea or ammonia and an operating temperature are measured, and a supply amount of the oxygen is adjusted in response to measurement values of the concentration and the operating temperature to control a corrosion rate and a reaction yield of urea; and
(C) a control method in which operating pressures of the plurality of processing units and their respective operating temperatures, a flow rate of carbon dioxide introduced as the raw material, an oxygen amount in the raw material carbon dioxide and a flow rate of ammonia introduced as the raw material are measured to calculate respective corrosion rates of the plurality of processing units and corrosion rates of the plurality of lines connecting the plurality of processing units, and a supply amount of the oxygen is adjusted thereby to control a corrosion rate and a reaction yield of urea.

4. The method for manufacturing urea according to claim 3, comprising, when the control methods (A), (B) and (C) are performed in this order:
determining, based on a corrosion rate in the control method (A), whether to increase the oxygen supply amount to the raw material carbon dioxide, not performing the control methods (B) and (C) when the oxygen supply amount is increased, and proceeding to the control method (B) when the oxygen supply amount is not increased;
when proceeding to the control method (B), determining, based on a corrosion rate in the control method (B), whether to increase the oxygen supply amount to the raw material carbon dioxide, and not performing the control method (C) when the oxygen supply amount is increased, and proceeding to the control method (C) when the oxygen supply amount is not increased; and
when proceeding to the control method (C), determining, based on a corrosion rate in the control method (C), whether to increase the oxygen supply amount to the raw material carbon dioxide, not performing further processes when the oxygen supply amount is increased, and, when the oxygen supply amount is not increased, determining whether to maintain the current oxygen supply amount to the raw material carbon dioxide or to decrease the oxygen supply amount to the raw material carbon dioxide, based on the respective corrosion rates in the control methods (A) to (C).
